(19) United States
(12) Patent Application Publication
(10) Pub. No.: US 2012/0207725 A1
(43) Pub. Date: Aug. 16, 2012

(54) MESENCHYMAL STEM CELL INCORPORATING A NUCLEOTIDE SEQUENCE CODING TGFB, AND USES THEREOF

(76) Inventors: Seok Goo Cho, Seoul (KR); Min Jung Park, Incheon (KR); Hyun Sil Park, Seoul (KR); Mi La Cho, Seoul (KR)

(21) Appl. No.: 13/503,390

(22) PCT Filed: Aug. 27, 2010
(86) PCT No.: PCT/KR10/05771

§ 371 Date: Apr. 23, 2012

(30) Foreign Application Priority Data
Oct. 23, 2009 (KR) 10-2009-0101194

(51) Int. Cl. A61K 35/12; A61P 9/00; A61P 7/06; A61P 19/02; A61P 1/00; A61P 3/10; A61P 25/00; A61P 17/06; A61K 35/28; A61P 31/12

(52) U.S. Cl. 424/93.21

(57) ABSTRACT

One or more specific examples of the present invention relate to a mesenchymal stem cell incorporating a nucleotide sequence coding TGFβ, and to the uses thereof.

### TECHNICAL FIELD

The present disclosure relates to mesenchymal stem cells introduced with a transforming growth factor beta (TGFβ)-encoding nucleotide sequence, and use thereof.

### BACKGROUND ART

Mesenchymal stem cells are a kind of adult stem cells present in the bone marrow with hemotopoietic stem cells, which are available from the bone marrow or umbilical cord blood, and are relatively easy to be separated and proliferated. Mesenchymal stem cells secrete a variety of water-soluble factors, and may differentiate into various mesoblastic cell lines (such as chondroblast, osteoblast, fibroblast, adipose cells) and tissues, so there have been endeavors to use mesenchymal stem cells in the treatment of tissue damage. Mesenchymal stem cells are known to have immune tolerance and suppression effects in transplant and autoimmunity models. Simultaneous regulation of immunity regulatory T cells and Th17 cells that lead to disease-causing autoimmune reactions are very significant in immune diseases, and cancer or other transplant rejection diseases.

Transforming growth factor beta (TGFβ) is a secreted protein present in three isoforms: TGFβ1, TGFβ2, and TGFβ3. TGFβ is expressed as large protein precursor, of which, TGFβ1 includes 390 amino acids, and TGFβ2 and TGFβ3 each include 412 amino acids. TGFβ has a pro-region called latency-associated peptide (LAP), which is an N-terminal signal peptide consisting of 20-30 amino acids required for secretion from cells, and a C-terminal region consisting of 112-114 amino acids that are released from the pro-region via protein cleavage and contribute to mature TGFβ molecules. The term TGFβ as used herein means to include a TGFβ precursor or a matured TGFβ.

Despite of the prior technologies, there still is a demand for a composition that increases CD4+CD25+Foxp3 regulatory T cells and at the same time decreases Th17 cells when administered to a subject suffering from an autoimmune disease caused by an autoantigen.

### DETAILED DESCRIPTION OF THE DISCLOSURE

### Technical Problem

An embodiment of the present disclosure provides a composition for treating an autoimmune disease of an individual organism.

Another embodiment of the present disclosure provides a composition that increases autoantigen-specific CD4+ CD25+ Foxp3+ regulatory T cells and reduces Th17 cells in an organism.

Another embodiment of the present disclosure provides a method of treating an autoimmune disease of an organism.

Another embodiment of the present disclosure provides a method for increasing self-antigen-specific CD4+ Foxp3+ regulatory T cells and reducing Th17 cells.

### Technical Solution

According to an aspect of the present disclosure, there is provided a composition for treating an autoimmune disease of an organism, the composition including a TGFβ-encoding mesenchymal stem cell introduced with a TGFβ-encoding nucleotide sequence, and a pharmaceutically acceptable carrier.

According to another aspect of the present disclosure, there is provided a composition for increasing self-antigen-specific CD4+ CD25+ Foxp3+ regulatory T cells and reducing Th17 cells, the composition including mesenchymal stem cells introduced with a TGFβ-encoding nucleotide sequence, and a pharmaceutically acceptable carrier.

According to another aspect of the present disclosure, there is provided a method of treating an autoimmune disease of an organism, the method including administering the above-described composition to the organism.

According to another aspect of the present disclosure, there is provided a method for increasing self-antigen-specific CD4+ CD25+ Foxp3+ regulatory T cells and reducing Th17 cells, the method including administering a pharmaceutical composition that includes mesenchymal stem cells introduced with a TGFβ-encoding nucleotide sequence, and a pharmaceutically acceptable carrier, to an organism.

### Advantageous Effects

The composition may effectively treat an autoimmune disease of an organism.

The composition for increasing autoantigen-specific CD4+ CD25+ Foxp3+ regulatory T cells and reducing Th17 cells in an organism may increase the autoantigen-specific CD4+ CD25+ Foxp3+ regulatory T cells and reduce the Th17 cells in the organism.

The treatment method may efficiently treat an autoimmune disease of an organism.

The method for increasing self-antigen-specific CD4+ Foxp3+ regulatory T cells and reducing Th17 cells may increase self-antigen-specific CD4+ Foxp3+ regulatory T cells and reduce Th17 cells in an organism.

### BRIEF DESCRIPTION OF THE DRAWINGS

FIG. 1 is a map of a pAdlox-eGFP TGFb vector including a nucleotide sequence (TGF-b) for encoding TGFβ1 of SEQ. ID No. 1;

FIG. 2 is a graph of arthritic indices in animals with collagen induced arthritis (CIA) over 15 weeks after an one-time intraperitoneal injection of bone marrow-derived mesenchymal stem cells or TGFβ gene-inserted, bone marrow-derived mesenchymal stem cells into the animals;

FIG. 3 is graphs of results of flow cytometry using a fluorescence activated cell sorter (FACS), illustrating degrees of differentiation into CD25 positive T cells when CD4+CD25− T cells separated from spleen cells of a normal mouse were co-incubated for 3 days with bone marrow-derived mesenchymal stem cells (+MSC) or TGFβ gene-inserted bone marrow-derived mesenchymal stem cells (+TGFb MSC); and

FIG. 4 presents results of analysis by flow cytometry using an FACS on differentiation of immune regulatory T cells (CD4+ Foxp3+ regulatory T cells, Treg) and IL-17-secreting T cells in the spleen cells of the animal model after incubation alone or co-incubation with type II collagen (CII) as a stimulating self-antigen for 3 days.

### BEST MODE

The present disclosure will be described in further detail with reference to the following examples. These examples are for illustrative purposes only and are not intended to limit the scope of the disclosure.

### EXAMPLE 1

(1) Separation of Mesenchymal Stem Cells

To differentiate mesenchymal stem cells, after removal of the skin and muscle of a 6-week-old DBA1J mouse, the thighbone and shinbone were removed from the mouse. Subsequently, a Roswell Park Memorial Institute (RPM I) medium containing a 0.3% bovine serum albumin (BSA) was injected into the bone using a 26G syringe to extract monocytes from the bone marrow. The extracted bone marrow monocytes were incubated in a Dulbecco's Modified Eagles Medium (DMEM) containing a 10% fetal bovine serum (FBS) at a 37° C., 5% CO₂ incubator. Once saturated over 5-7 days, the incubated product was sub-cultured, while morphological changes in the cells were microscopically observed during a time interval. After 10 or more subcultures, flow cytometry was conducted using a CD marker to investigate whether a cellular surface antigen representing a characteristic of stem cells was expressed in the isolated mesenchymal stem cells or not. The incubated cells tested positive for mesenchymal cellular surface antigens CD29, CD44, and Sca-1, but tested negative for hematopoietic stem cell surface antigens CD34 and CD45.

(2) Introduction of TGFβ Gene into Mesenchymal Stem Cells

Adenoviruses are able to express an abundance of foreign genes by efficient cellular infections, and thus, are frequently used as a gene delivery vehicle that delivers therapeutic genes for various types of diseases into the body. To prepare and separate recombinant adenoviruses, a pAdlox-eGFP TGFb vector was added in a concentration of 2×10⁹/ml into the medium to prepare a virus stock.

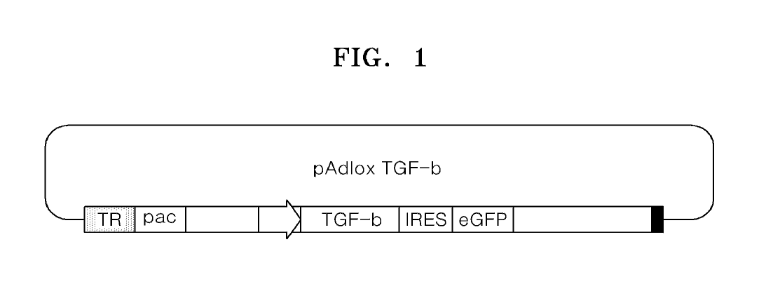

FIG. 1 presents a map of the pAdlox-eGFP TGFb vector, which includes a nucleotide sequence (TGF-b) encoding TGFβ1 of SEQ ID No. 1. The pAdlox-eGFP TGFb vector of FIG. 1 is a vector system expressing a TGFβ1 gene, in which TR, pac, IRES, and eGFP in FIG. 1 are essential components for virus packaging.

After dilution of the virus stock with a serum-free DMEM medium, the mesenchymal stem cells separated from the DBA1J mouse were infected with the pAdlox-eGFP TGFβ vector at a multiplicity of infection (MOI) of 100. After a medium change with a DMEM containing 10% FBS, the infected cells in the DMEM were cultured in a 37° C., 5% CO₂ incubator for about 24 hours and then collected. An expression of the TGFβ introduced into the mesenchymal stem cells was identified through an expression of eGFP by fluorescent microscopy and flow cytometry, and a concentration of the TGFβ was measured by immunoenzymetric assay.

(3) Measurement of Arthritis Treatment Efficacy of TGFβ-Introduced Mesenchymal Stem Cells in Mice by Intraperitoneal Administration

(3.1) Preparation of Animal Model and Administration

An animal model with collagen-induced arthritis (CIA) was prepared, and TGFβ gene-introduced mesenchymal stem cells were administered to the animal model as follows:

Six to seven-week-old male DBA-1 mice were used as test animals. Type II collagen (CII) was dissolved in a 0.1 N acetic acid solution to a concentration of 4 mg/ml, and was then dialyzed using a dialysis buffer (50 mM Tris, 0.2N NaCl). This dialysis product was mixed with a complete Freund's adjuvant (CFA) (available from Chondrex) containing M. tuberculosis in an equal ratio, followed by subcutaneous injection at the base of the tail so that 100 μl of the immunogen (i.e., 100 μl/100 μg) was injected in each mouse (1^{st} injection). Two weeks after the 1^{st} injection, the same type II collagen (CII) was mixed with an incomplete Freund's adjuvant (IFA) (available from Chondrex) in an equal ratio, followed by injection of the same amount at one of the two hind legs (2^{nd} injection).

Seven weeks after the 2^{nd} injection, 200 μl of mesenchymal stem cells (a control group) or TGFβ gene-inserted mesenchymal stem cells, 1×10⁶/200 μl was injected into the peritoneal cavity.

Each experimental group consisted of six mice. After an in vitro arthritis test over 15 weeks, the mice were killed at an appropriate time for a significant arthritic index, and changes in immunocytes of the spleen associated with activation of arthritis were observed.

(3.2) Rheumatoid Arthritis Treatment Activity of TGFβ Gene-Inserted Mesenchymal Stem Cells in CIA Animals

Three weeks after the 1^{st} injection, the seriousness of the arthritis was evaluated three times per week for 10 weeks by three observers who were unaware of the experiment. The arthritis evaluation was performed using an average arthritic index of Rossoliniec et al. (Wooley J. Exp. Med. 154 (3): 688-700), in which symptoms at the three remaining legs of each mouse, excluding the one hind leg at which CII and CFA was injected in the second injection, were evaluated as a score by three observers based on the following criteria. A sum of the scores from the three observers was divided by three to obtain an average. The score for the arthritis evaluation and criteria are as follows.

Score 0: Neither edema nor swelling was observed.

Score 1: Minor local edema and redness occurred in the foot or ankle joint.

Score 2: Minor edema and redness occurred over from the ankle joints to metatarsals.

Score 3: Moderate edema and redness occurred over from the ankle joint to metatarsals.

Score 4: Edema and redness occurred over the entire leg.

The largest arthritic index each observer may assign to each mouse is a score of 4, and thus, each mouse may have a largest arthritic index of 16, which is the sum of the scores from the three observers.

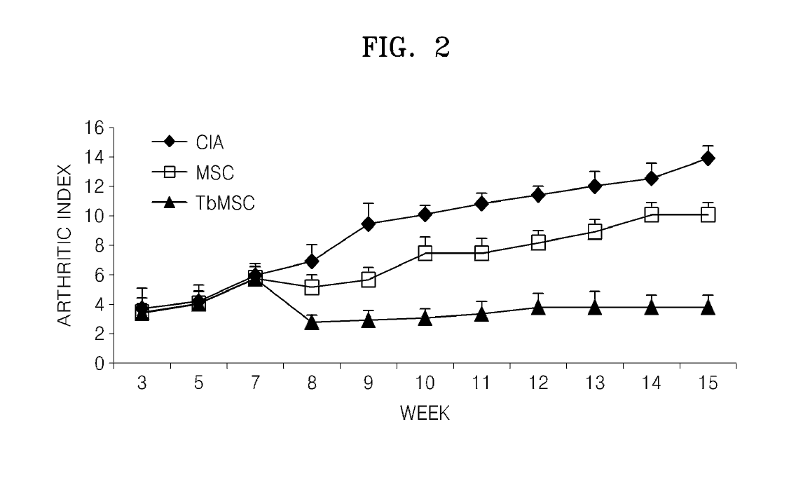

In the test mice injected with the TGFβ gene-inserted mesenchymal stem cells the artistic indices were found to gradually reduce. On the other hand, in the animals with the CIA and the animals in which mesenchymal stem cells were not injected, common clinical arthritis symptoms occurred, with an increasing difference with respect to time from the test animals injected with the TGFβ gene-inserted mesenchymal stem cells (see FIG. 2).

FIG. 2 is a graph of arthritic indices obtained with the animals with CIA through observation for 15 weeks from the 1^{st} intraperitoneal injection of the mesenchymal stem cells or TGFβ gene-inserted mesenchymal stem cells. In FIG. 2, “CIA” indicates an animal model group with CIA, “MSC” indicates an animal group of the CIA animal model into which the mesenchymal stem cells were injected, and “TbMSC” indicates an animal group of the CIA animal model into which the TGFβ gene-inserted mesenchymal stem cells were injected.

(4) Induction of Regulatory CD4+ T Cells and Suppression of Th17 Cells by TGFβ Gene-Inserted Mesenchymal Stem Cells

To identify a treatment mechanism of the TGFβ gene-inserted mesenchymal is stem cells for rheumatoid arthritis, an immune system that is induced or suppressed by the TGFβ gene-inserted mesenchymal stem cells was investigated.

(4.1) Induction of Regulatory CD4+ T Cells by TGFβ Gene-Inserted Mesenchymal Stem Cells

After isolated from the killed CIA animals, the spleen cells were cultured alone in a medium in a 37° C., 5% CO₂ incubator or were co-cultured along with the type II collagen (CII) in a concentration of 40 μg/ml in a 37° C., 5% CO₂ incubator for 3 days, followed by flow cytometry using a fluorescence-activated cell sorter (FACS) to observe Foxp3-expressing cells and changes in Th17 cells.

As a result, compared with the CIA animal group and the control group with the mesenchymal stem cells, the animal group with the TGFβ gene-inserted mesenchymal stem cells was found to include increased CD4+ CD25+ Foxp3+ regulatory T cells and reduced Th17 cells in the isolated spleen when stimulated with the type II collagen (CII), relative to when cultured alone in the medium. In conclusion, CD4+ CD25+ Foxp3+ regulatory T cells specific to the type II collagen (CII) were generated, suppressing overproliferation of chronic inflammatory IL-17 producing T cells (Th17 cells) associated with a cause of rheumatoid arthritis. This leads to balance between inflammatory cytokine and anti-inflammatory cytokine, indicating that a progress of rheumatoid arthritis may be suppressed, and effective treatment of rheumatoid arthritis.

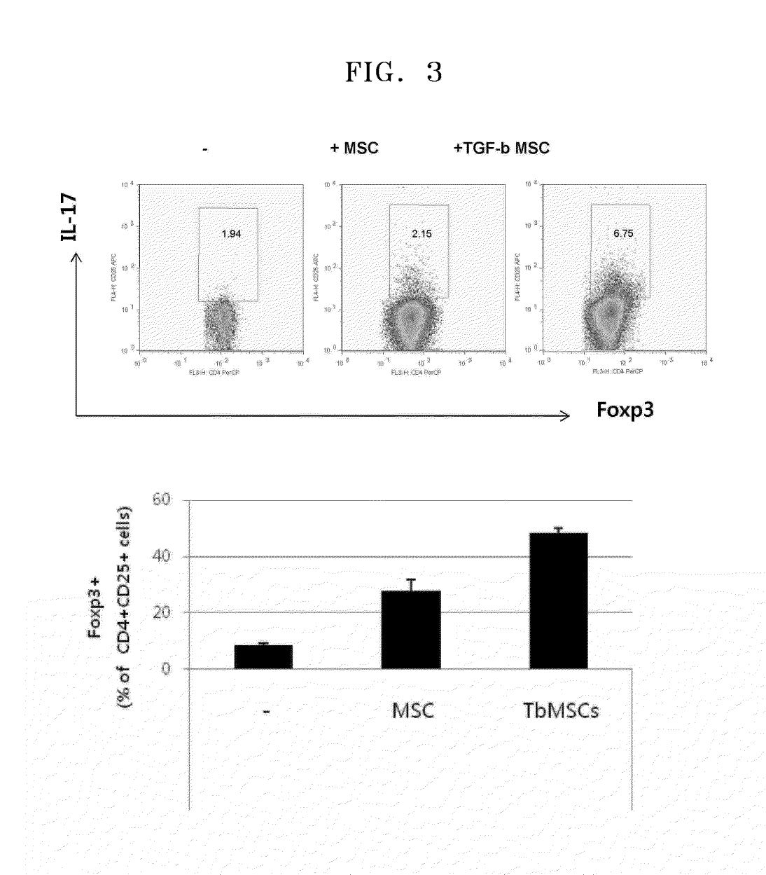

FIG. 3 is graphs of results of flow cytometry using an FACS, illustrating degrees of differentiation into CD25 positive T cells when CD4+CD25− T cells isolated from spleen cells of a normal mouse were co-incubated for 3 days with bone marrow-derived mesenchymal stem cells (+MSC) or TGFβ gene-inserted bone marrow-derived mesenchymal stem cells (+TGFb MSC).

As illustrated in FIG. 3, when the bone marrow-derived mesenchymal stem cells (+TGFb MSC) and the spleen cells were co-cultured, a percentage of the CD4+CD25+Foxp3+ regulatory T cells was increased than otherwise (FIG. 3).

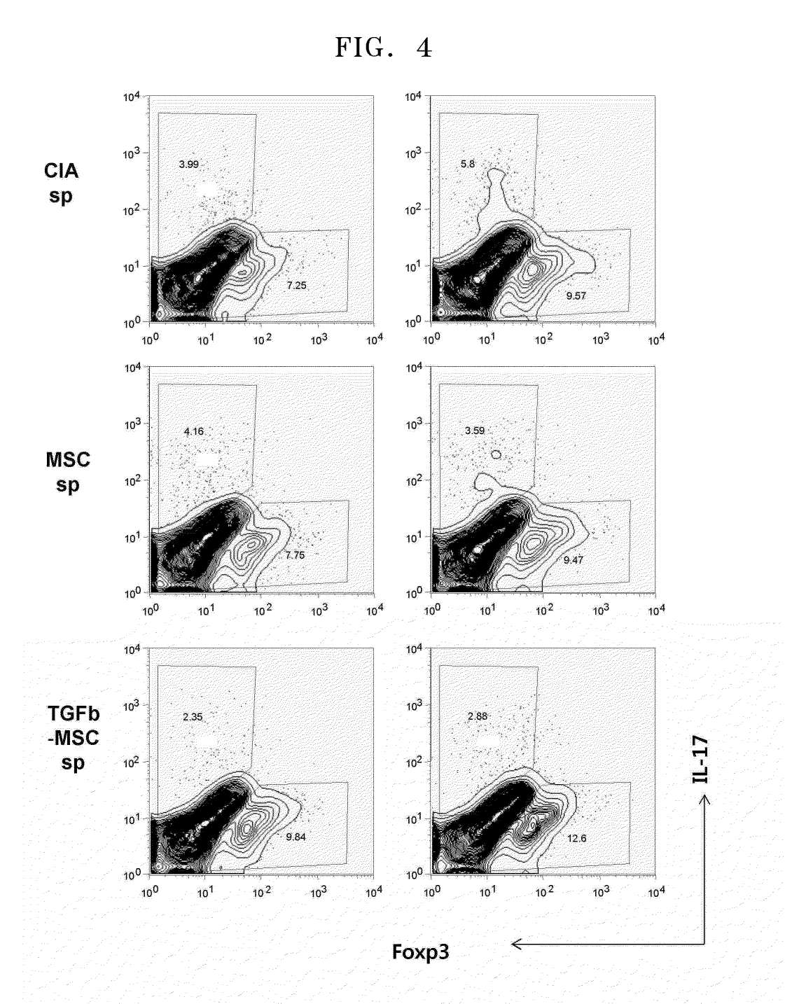

FIG. 4 presents results of analysis by flow cytometry using an FACS on differentiation of immune regulatory T cells (CD4+ Foxp3+ regulatory T cells, Treg) and IL-17 secreting T cells in the spleen cells of the animal model after incubation alone or co-incubation with 40 μg/ml of the type II collagen (CII) as a stimulating self-antigen for 3 days.

In FIG. 4, CIA, MSC, and TGFb-MSC indicate results from the spleen cells isolated from an arthritis animal model, those from the spleen cells of an arthritis animal model to which the mesenchymal stem cells were administered, and those from the spleen cells of an arthritis animal model to which TGFβ-inserted mesenchymal stem cells were administered, respectively; and Nil and CII indicate those from incubation alone and co-incubation with CII, respectively. As shown in FIG. 4, when the spleen cells isolated from the arthritis animal model administered with the TGFβ-inserted mesenchymal stem cells were co-cultured with the self-antigen CII, the CD4+CD25+foxp3+regulatory T cells were increased, but Th17 cells were reduced, as compared with when the spleen cells isolated from arthritis animal model administered only with the mesenchymal stem cells were co-cultured with the self-antigen CII.

As in the results described above, the TGFβ-inserted mesenchymal stem cells are found to be effective in the treatment of autoimmune diseases caused from an excessive immune reaction against the self-antigen.

### Mode of the Disclosure

An aspect of the present disclosure provides a composition for treating an autoimmune disease of an organism, the composition including a TGFβ-encoding mesenchymal stem cell introduced with a TGFβ-encoding nucleotide sequence, and a pharmaceutically acceptable carrier.

Transforming growth factor beta (TGFβ) is a secreted protein present in three isoforms: TGFβ1, TGFβ2, and TGFβ3. TGFβ is expressed as a large protein precursor, and in particular, TGFβ1 includes 390 amino acids, TGFβ2 and TGFβ3 each include 412 amino acids. TGFβ has a pro-region called latency-associated peptide (LAP), which is an N-terminal signal peptide consisting of 20-30 amino acids required for secretion from cells, and a C-terminal region consisting of 112-114 amino acids that are released from the pro-region via protein cleavage and contribute to mature TGFβ molecules. The term TGFβ as used herein means to include a TGFβ precursor or a matured TGFβ. For example, TGFβ may be a TGFβ1 precursor or a matured TGFβ1. The TGFβ-encoding nucleotide sequence may encode an amino acid sequence of SEQ ID No. 2, i.e., an amino acid sequence of TGFβ1. The TGFβ-encoding nucleotide sequence may have a nucleotide sequence of SEQ. ID No. 1, i.e., a nucleotide sequence encoding TGFβ1.

The TGFβ-encoding nucleotide sequence may be introduced into mesenchymal stem cells by a known method in the art. In some embodiments, the TGFβ-encoding nucleotide sequence may be introduced directly or using a vector. Methods of introducing nucleic acid sequences into cells are widely known. For example, a nucleic acid sequence may be introduced by electroporation or using calcium phosphate, a gene gun, or liposome. A nucleic acid sequence may be introduced via a viral carrier. TGFβ-encoding nucleotide sequence may be present by being integrated with a cellular genome, or may be in a cell separate from the genome.

As used herein, the term “vector” means a nucleic acid molecule able to carry other nucleic acids. Considering that the nucleic acid sequence mediates introduction of a specific gene, the vector used herein is construed to be interchangeable with a nucleic acid construct, or a cassette. Examples of the vector are a plasmid vector and a virus-derived vector. A plasmid is a circular double-stranded DNA molecule linkable with another DNA. Non-limiting examples of the vector used in the present disclosure are a plasmid expression vector, a virus expression vector (for example, SV40, replication-defective retrovirus, adenovirus, and adeno-associated virus (AAV)), and other viral vectors having equivalent functions to these vectors.

The TGFβ-encoding nucleotide sequence may be introduced by, for example, an adenovirus-associated vector. An adenovirus-associated vector refers to a vector using an AAV that is a small virus causing infection to humans and other primate species. AAV is not known to cause disease and consequently the virus causes a very mild immune response. AAV can infect both dividing and non-dividing cells and integrate into the genome of the host cells. These features make AAV a very attractive candidate for creating viral vectors for gene therapy. The adenovirus-associated vector may be a pAdlox-eGFP TGFb vector having a nucleotide sequence of SEQ ID No. 1.

As used herein, the “mesenchymal stem cells” means multipotent stem cells able to differentiate into a variety of cell types. For example, the mesenchymal stem cells may differentiate into osteoblasts, adipocytes, myoblasts, and chondrocytes. Normally, mesenchymal stem cells have at least one of the following characteristics: the ability of asynchronous replication in which two daughter cells may have different phenotypes after division, or the ability of symmetric replication; and the ability of clonal regeneration of a tissue in which mesenchymal stem cells are, for example, non-hematopoietic cells of the bone marrow. The mesenchymal stem cells may include bone marrow-derived mesenchymal stem cells or fat-derived mesenchymal stem cells. The “bone marrow-derived mesenchymal stem cells” may include mesenchymal stem cells separated from the bone marrow or bone marrow-derived mesenchymal stem cells obtained by culturing the separated mesenchymal stem cells. The “fat-derived mesenchymal stem cells” may include mesenchymal stem cells separated from a fat tissue, or fat marrow-derived mesenchymal stem cells obtained by culturing the separated mesenchymal stem cells. Separating mesenchymal stem cells is widely known in the art. For example, bone marrow-derived mesenchymal stem cells may be obtained by a known method (Pittenger et al.(1999) Science 284(5411); Liechty et al.(2000) Nature Medicine 6; 1282-1286). Separation of bone marrow-derived mesenchymal stem cells may involve, for example, separating bone marrow cells from the thighbone or shinbone of a mouse, subsequent sub-culturing ten times or more in a DMEM medium, for example, in a 37° C., 5% CO₂ incubator, and analyzing a surface antigen by flow cytometry. A method of culturing bone marrow-derived mesenchymal stem cells is known. For example, the separated mesenchymal stem cells may be cultured in an IMDB medium or a DMEM medium at about 37° C.

As used herein, the “pharmaceutically acceptable carrier” may be a diluent, an excipient, a disintegrant, a binder, or a lubricant, but is not limited thereto. For example, the pharmaceutically acceptable carrier may contain a medium for culturing mesenchymal stem cells, such as bone marrow-derived mesenchymal stem cells, injectable water, and a buffer, but is not limited thereto. The buffer may be phosphate buffered saline (PBS). The pharmaceutically acceptable carrier may be a diluent including at least one selected from the group consisting of lactose, corn starch, soybean oil, amorphous cellulose, and mannitol.

The TGFβ-encoding nucleotide sequence may be introduced into the mesenchymal stem cells to be expressible. For example, the nucleotide sequence may be linked to be operable with a promoter and a regulatory site such as polyadenylation sites, so that the nucleotide sequence may be expressible within the mesenchymal stem cells. Thus, the TGFβ-encoding nucleotide sequence in the mesenchymal stem cells may be involved in overexpressing TGFβ in the mesenchymal stem cells as compared with mesenchymal stem cells into which no TGFβ-encoding nucleotide sequence is introduced. For example, a degree of the over-expression may be about 5% or greater, 10% or greater, or 15% or greater based on the amount of an active protein, as compared with that in the mesenchymal stem cells into which no TGFβ-encoding nucleotide sequence is introduced.

The organism may be a mammal. The mammal may be, for example, a human or a non-human primate. In some embodiments, the organism may be a human, a monkey, a dog, a cat, a cow, or a mouse.

As used herein, the “autoimmune disease” means a disease caused by an excessive immune reaction of an organism to a normal substance and/or tissues in the organism. For example, the autoimmune disease may be selected from the group consisting of acute disseminated encephalomyelitis (ADEM), Addison's disease, autoimmune hemolytic anemia, autoimmune hepatitis, chronic obstructive pulmonary disease (COPD), Crohn's disease, diabetes mellitus type 1, idiopathic thrombocytopenic purpura, lupus erythematosus, multiple sclerosis (MS), pemphigus vulgaris, pernicious anaemia, psoriasis, psoriatic arthritis, rheumatoid arthritis, sjogren's syndrome, ulcerative colitis, and vasculitis.

In the composition for treatment of autoimmune disease, the TGFβ-encoding nucleotide sequence-inserted mesenchymal stem cells may further increase self-antigen-specific CD4+ CD25+ Foxp3+ regulatory T cells and reduce Th17 cells, as compared with the mesenchymal stem cells into which no TGFβ-encoding nucleotide sequence is inserted. Thus, the TGFβ-encoding nucleotide sequence-inserted mesenchymal stem cells increase self-antigen-specific CD4+ CD25+ Foxp3+ regulatory T cells and at the same time reduce Th17 cells, thus suppressing a cause of an autoimmune-derived disease.

For example, the self-antigen may be selected from the group consisting of a collagen type II protein, smooth muscle actin, bullous pemphigoid antigens 1 and 2, a transglutaminase, elastin, a basement membrane collagen type IV protein, ganglioside, desmoglein 3, p62, sp100, a rheumatoid factor, and a topoisomerase.

As used herein, the term “treatment” refers to relieve, treat, improve, or further prevent a disease of an organism.

The CD4+ CD25+ Foxp3+ regulatory T cells are regulatory T cells expressing CD4, CD25 and Foxp3 (CD4+ CD25+ Foxp3+ regulatory T cell or Treg). Regulatory T cells are a component of the immune system that suppress immune responses of other cells. This is an important “self-check” built into the immune system to prevent excessive reactions. These regulatory T cells are involved in shutting down immune responses after they have successfully eliminated invading organisms, and also in regulating potential attack of self tissues (autoimmunity). CD4+ CD25+ Foxp3+ regulatory T cells are called “naturally-occurring” regulatory cells to distinguish them from “suppressor” T cell populations that are generated in vitro. Self-antigen-specific CD4+ CD25+ Foxp3+ regulatory T cells may suppress immune response of cells including self-antigens described above, i.e., CD4, CD25 and Foxp3. Regulatory T cells are defined by an expression of the forkhead family transcription factor FOXP3 (abbreviation for “forkhead box p3”). An expression of FOXP3 is required for regulatory T cell development and appears to control a genetic program specifying the fate of the cell. CD4+ CD25+ Foxp3+ regulatory T cells express FOXP3, CD4, and IL-2 receptor alpha chain (CD25).

T helper 17 cells (Th17 cells) are a subset of T helper cells producing interleukin 17 (IL-17). Excessive amounts of the Th17 cell are thought to involve in an onset of autoimmune disease. Th17 cells are thought to play a role in inflammation and tissue injury in inflammatory conditions, and can cause severe autoimmune diseases. TGFβ, IL-6, IL-21, and IL-23 are known to be involved in generation of Th17 cells in humans and mice (Dong C (May 2008), Nat. Rev. Immunol. 8(5); 337-48; Manel N et al. (June 2008), Nat. Immunol. 9(6); 641-9).

Therefore, with use of the composition of the present disclosure, the self-antigen-specific CD4+ CD25+ Foxp3+ regulatory T cells are increased to suppress immune responses to excessive self-antigens, and the Th17 cells involved in autoimmune diseases are reduced to significantly treat autoimmune diseases.

According to another aspect of the present disclosure, there is provided a composition for increasing self-antigen-specific CD4+ CD25+ Foxp3+ regulatory T cells and reducing Th17 cells, the composition including mesenchymal stem cells introduced with a TGFβ-encoding nucleotide sequence, and a pharmaceutically acceptable carrier.

According to another aspect of the present disclosure, there is provided a method of treating an autoimmune disease of an organism, the method including administering the above-described therapeutic composition for an autoimmune disease to the organism.

The composition may be administered to the organism by any method known in the art, for example, orally or non-orally. Non-limiting examples of non-oral administration are intraperitoneal, intravenous, intrathecal, intramuscular, subcutaneous, intradermic, intranasal, intramucosal, and intravaginal administration.

An administration amount of the composition may be a “therapeutically effective amount” that is sufficient to treat autoimmune disease. The therapeutically effective amount may be sufficient to relieve, improve, treat, or prevent autoimmune disease. The administration amount of the composition may be appropriately chosen depending on the type and seriousness of the autoimmune disease, body weight, age, and gender of the patient. The administration amount may be about 1×10⁴ cell/kg of body weight to about 1×10⁶cells/kg of body weight, and in some embodiments, may be from about 5×10⁴ cells/kg of body weight to about 1×10⁶cells/kg of body weight.

In the treatment method of the present disclosure, the “composition for treating an autoimmune disease” and “organism” are the same as those described above.

According to another aspect of the present disclosure, there is provided a method for increasing self-antigen-specific CD4+ CD25+ Foxp3+ regulatory T cells and reducing Th17 cells, the method including administering a pharmaceutical composition that includes mesenchymal stem cells introduced with a TGFβ-encoding nucleotide sequence, and a pharmaceutically acceptable carrier, to an organism.

In the method, the pharmaceutical composition may be administered to the organism by any method known in the art, for example, orally or non-orally. Non-limiting examples of non-oral administration are intraperitoneal, intravenous, intrathecal, intramuscular, subcutaneous, intradermic, intranasal, intramucosal, and intravaginal administration.

An administration amount of the composition may be an amount which is sufficient to increase the self-antigen-specific CD4+ CD25+ Foxp3+ regulatory T cells and reduce the Th17 cells, relative to before administration of the composition. The administration amount of the composition may be appropriately chosen depending on the type and seriousness of the autoimmune disease, body weight, age, and gender of the patient. The administration amount may be about 1×10⁴ cell/kg of body weight to about 1×10⁶cells/kg of body weight, and in some embodiments, may be from about 5×10⁴ cells/kg of body weight to about 1×10⁶ cells/kg of body weight.

In the method, the “pharmaceutical composition”, “organism”, “self-antigen-specific CD4+ CD25+ Foxp3+ regulatory T cells”, and “Th17 cells” are the same as those described above.

### SEQUENCE LIST FREE TEXT

The specification is described with reference to the SEQ. ID Nos in a sequence list appended therewith. The sequence list appended herewith is incorporated herein in its entirety by reference.

1. A composition for treating an autoimmune disease of an organism, the composition comprising:
a mesenchymal stem cell introduced with a TGFβ-encoding nucleotide sequence, and a pharmaceutically acceptable carrier.

2. The composition of claim 1, wherein the TGFβ-encoding nucleotide sequence encodes an amino acid sequence of SEQ ID No. 2.

3. The composition of claim 2, wherein the TGFβ-encoding nucleotide sequence has a nucleotide sequence of SEQ ID No. 1.

4. The composition of claim 1, wherein the TGFβ-encoding nucleotide sequence is introduced by an adenovirus associated vector.

5. The composition of claim 1, wherein the mesenchymal stem cells are bone marrow-derived mesenchymal stem cells or fat-derived mesenchymal stem cells.

6. The composition of claim 1, wherein the mesenchymal stem cell introduced with the TGFβ-encoding nucleotide sequence is able to overexpress TGFβ in the organism, relative to mesenchymal stem cells into which no TGFβ-encoding nucleotide sequence is introduced.

7. The composition of claim 1, wherein the autoimmune disease is selected from the group consisting of acute disseminated encephalomyelitis (ADEM), Addison's disease, autoimmune hemolytic anemia, autoimmune hepatitis, chronic obstructive pulmonary disease (COPD), Crohn's disease, diabetes mellitus type 1, idiopathic thrombocytopenic purpura, Lupus erythematosus, multiple sclerosis (MS), pemphigus vulgaris, pernicious anemia, psoriasis, psoriatic arthritis, rheumatoid arthritis, sjogren's syndrome, ulcerative colitis, and vasculitis.

8. The composition of claim 1, wherein the mesenchymal stem cells are bone marrow-derived mesenchymal stem cells, and the mesenchymal stem cell introduced with the TGFβ-encoding nucleotide sequence increases self-antigen-specific CD4+ CD35+ Foxp3+ regulatory T cells, and reduces Th17 cells, as compared with bone marrow-derived mesenchymal stem cells into which no TGFβ-encoding nucleotide sequence is introduced.

9. The composition of claim 1, wherein the organism is a mammal

10. The composition of claim 9, wherein the mammal is a human or a mouse.

11. A method of treating an autoimmune disease of an organism, the method comprising administering the composition of claim 1 to the organism.

12. A method for increasing self-antigen-specific CD4+ CD25+ Foxp3+ regulatory T cells and reducing Th17 cells, the method comprising administering a pharmaceutical composition that comprises a mesenchymal stem cell introduced with a TGFβ-encoding nucleotide sequence, and a pharmaceutically acceptable carrier, to an organism.

13. The method of claim 12, wherein the organism is a non-human mammal

14. The method of claim 11, wherein the organism is a non-human mammal

15. The method of claim 11, wherein the TGFβ-encoding nucleotide sequence encodes an amino acid sequence of SEQ ID No. 2.

16. The method of claim 11, wherein the TGFβ-encoding nucleotide sequence has a nucleotide sequence of SEQ ID No. 1.

17. The method of claim 12, wherein the TGFβ-encoding nucleotide sequence encodes an amino acid sequence of SEQ ID No. 2.

18. The method of claim 12, wherein the TGFβ-encoding nucleotide sequence has a nucleotide sequence of SEQ ID No. 1.


SEQUENCE LISTING (2 sequences)

SEQ ID NO: 1 - DNA, 1173 bp, Homo sapiens
atgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctga

SEQ ID NO: 2 - protein, 390 aa, Homo sapiens
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS